(19) United States
(12) Patent Application Publication
(10) Pub. No.: US 2015/0185233 A1
(43) Pub. Date: Jul. 2, 2015

(54) METHOD AND APPARATUS FOR SEPARATING PLASMA FROM BLOOD FOR BILIRUBIN LEVEL ESTIMATION

(71) Applicant: KONINKLIJKE PHILIPS N.V., EINDHOVEN (NL)

(72) Inventors: Anil Shivram Raiker, Goa (IN); Ravindra Bhat, Bangalore (IN); Shrutin Ulman, Bangalore (IN)

(73) Assignee: KONINKLIJKE PHILIPS N.V. a corporation

(21) Appl. No.: 14/418,497

(22) PCT Filed: Jul. 17, 2013
(86) PCT No.: PCT/IB2013/055874

§ 371 Date: Jan. 30, 2015

(30) Foreign Application Priority Data
Aug. 8, 2012 (IN) 3258/CHE/2012

(51) Int. Cl. G01N 33/72; G01N 1/28; G01N 21/05; A61B 5/15; G01N 21/25

(52) U.S. Cl. CPC G01N 33/728; A61B 5/150755; G01N 21/25; G01N 21/05; G01N 1/28; G01N 2201/061; G01N 2201/02

(57) ABSTRACT

The present disclosure pertains to a system for separating plasma and/or serum from blood. The system is configured to separate plasma and/or serum from between about 20 and about 50 μl of blood. Separating plasma and/or serum from that amount of blood may be useful during a bilirubin level estimation in newborn babies. The system alleviates the need to centrifuge a sample of blood to separate the blood plasma and/or serum. The system is configured such that the separated serum is held by the system during an optical analysis to estimate bilirubin levels, thus eliminating the need to transfer the serum sample to a cuvette for analysis. In some embodiments, the system comprises a cartridge body, a filter, a serum pathway, an analysis port, a negative pressure source, a suction connector port, and/or other components.

The present disclosure pertains to a system and method for separating plasma and/or serum from blood.

It is well known to extract blood plasma and/or serum by separating blood cells. Different components of blood have different densities. Different components of blood, in particular blood cells, have different particle sizes. Suitably configured filters may capture, block, and/or pass particular components within blood. Separating blood cells and blood plasma and/or serum is commonly performed with a centrifuge.

Some newborn babies suffer from jaundice (hyperbilirubinemia). Although most newborns with jaundice are otherwise healthy, they must be monitored and treated, if necessary, because bilirubin is potentially toxic to the central nervous system. Bilirubin accumulates in the blood plasma and/or serum. To monitor bilirubin levels, it is well known to extract plasma from blood by separating blood cells, for example, through centrifugal force. Bilirubin levels are commonly estimated by optically analyzing blood plasma and/or serum separated using a centrifuge. The separated blood serum is placed in a cuvette, then in a spectroscope for analysis. As described above, the method for estimating bilirubin levels commonly involves multiple steps performed at various locations by multiple staff members within a medical facility. Separating blood plasma and/or serum often takes up to several hours at many medical facilities. In some countries (e.g., India and China), medical facilities lack the equipment necessary (e.g., centrifuges) to separate the blood plasma and/or serum, further increasing the complexity and time necessary for analysis because the sample requires testing services from an external facility. The current method for estimating bilirubin levels often requires an amount of blood of one milliliter (1 ml) or more.

Accordingly, one or more aspects of the present disclosure relate to a system for separating plasma and/or serum from blood. The plasma and/or serum separation system comprises a filter, a serum pathway, an analysis port, and a pressure source. The filter is configured to separate blood plasma and/or serum from a quantity of blood. The filter has an entry side and an exit side. The serum pathway is configured to collect the separated blood plasma and/or serum at the exit side of the filter. The analysis port, disposed in the serum pathway, is configured to hold a quantity of plasma and/or serum during a plasma and/or serum analysis. The analysis port is further configured to provide an optical path for radiation to pass through the plasma and/or serum during the plasma and/or serum analysis. The pressure source is configured to communicate with the serum pathway such that at least a portion of the blood plasma and/or serum on the exit side of the filter is directed into the analysis port.

Yet another aspect of the present disclosure relates to a method for separating plasma and/or serum from blood with a plasma and/or serum separation system. The plasma and/or serum separation system comprises a filter, a serum pathway, an analysis port, and a pressure source. The method comprises separating blood plasma and/or serum from a quantity of blood with the filter, wherein the filter has an entry side and an exit side; collecting the separated blood plasma and/or serum at the exit side of the filter with the serum pathway; holding a quantity of plasma and/or serum during a plasma and/or serum analysis with the analysis port, wherein the analysis port is disposed in the serum pathway, and wherein the analysis port is further configured to provide an optical path for radiation to pass through the plasma and/or serum during the plasma and/or serum analysis; and generating a pressure, with the pressure source, in the serum pathway such that at least a portion of the blood plasma and/or serum on the exit side of the filter is directed into the analysis port.

Still another aspect of the present disclosure relates to a system for separating plasma and/or serum from blood. The plasma and/or serum separation system comprises means to separate blood plasma and/or serum from a quantity of blood, wherein the means to separate has an entry side and an exit side; means to convey blood plasma and/or serum, the means to convey configured collect the separated blood plasma and/or serum at the exit side of the means to separate; means, disposed in the means to convey, to hold a quantity of plasma and/or serum during a plasma and/or serum analysis, wherein the means to hold is further configured to provide an optical path for radiation to pass through the plasma and/or serum during the plasma and/or serum analysis; and means to generate a pressure in the means to convey, the means to generate configured to communicate with the means to convey such that at least a portion of the blood plasma and/or serum on the exit side of the means to separate is directed into the means to hold.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

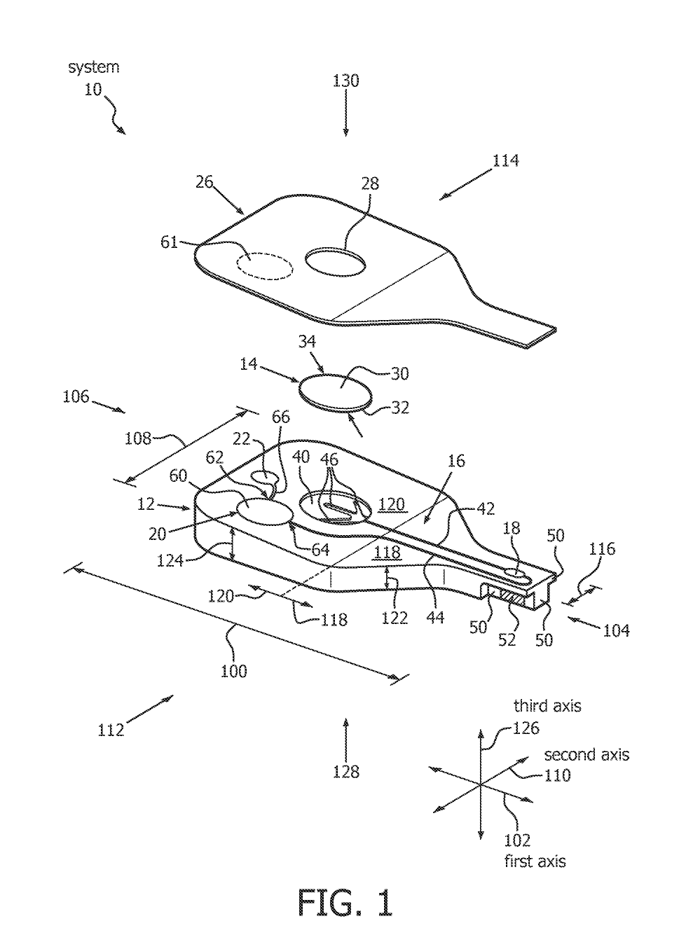

FIG. 1 is a partially exploded view of a blood plasma and/or serum separation system;

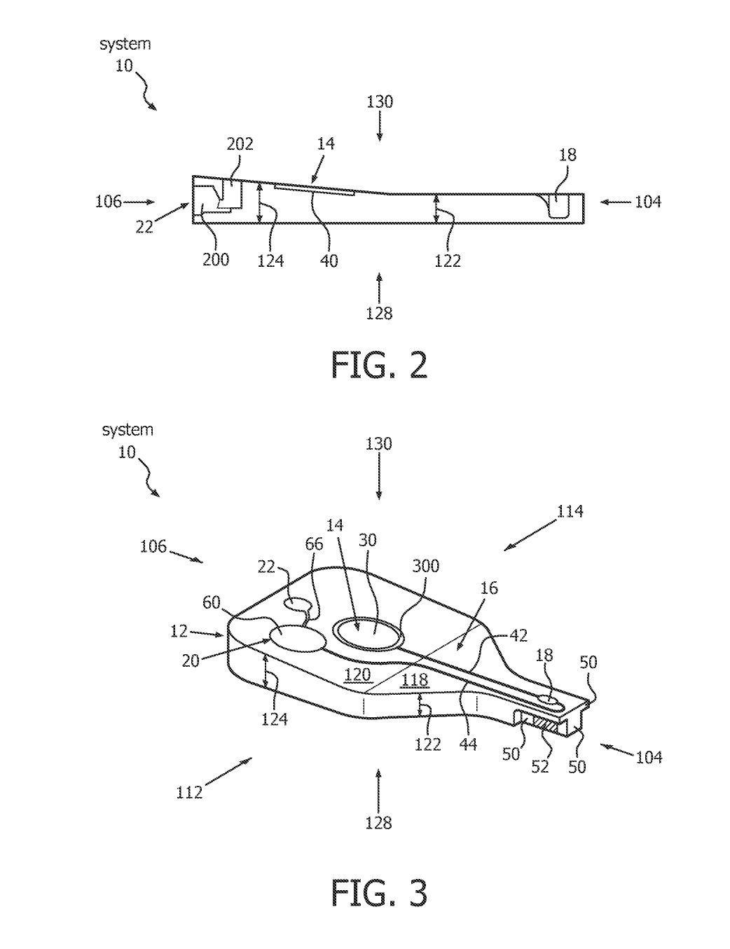

FIG. 2 is a sectional view of the blood plasma and/or serum separation system;

FIG. 3 is an assembled view of the blood plasma and/or serum separation system; and

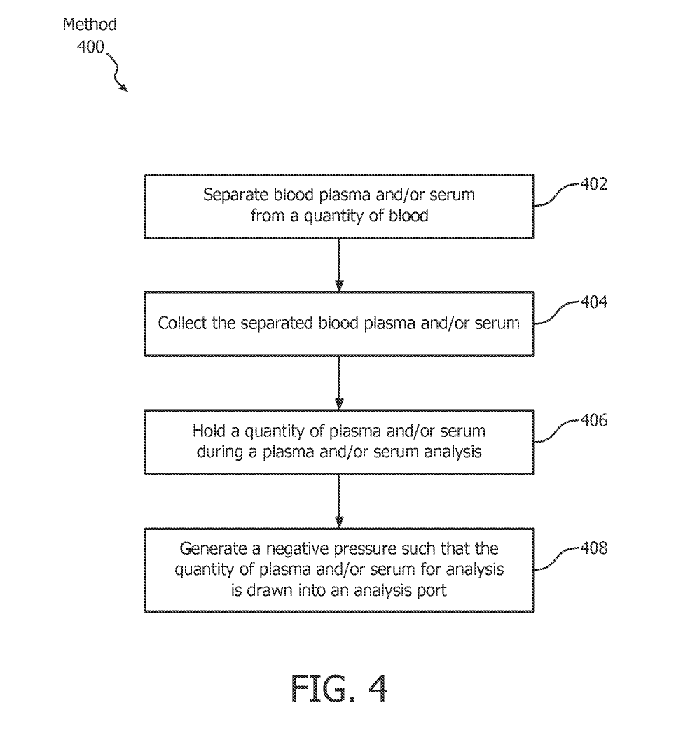

FIG. 4 illustrates a method of separating blood plasma and/or serum from blood.

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

FIG. 1 illustrates a partially exploded view of an example embodiment of a system 10 configured to separate plasma and/or serum from blood. System 10 is configured to separate plasma and/or serum from blood wherein the amount of blood is between about 20 and about 50 microliters (μl) of blood. Separating plasma and/or serum from that amount of blood may be useful during a bilirubin level estimation in newborn babies, for example. System 10 may be configured such that the steps required to estimate a bilirubin level are simplified, require less time, and require less blood compared to current methods. System 10 alleviates the need to centrifuge a sample of blood to separate the blood plasma and/or serum. System 10 is configured such that the separated serum is held by system 10 during optical and/or other analysis to estimate bilirubin levels. System 10 thus eliminates the need to transfer the serum sample to a cuvette and/or other holder for analysis. In some embodiments, system 10 comprises a cartridge body 12, a filter 14, a serum pathway 16, an analysis port 18, a negative pressure source 20, a suction connector port 22, and/or other components.

Cartridge body 12 is configured to house filter 14, serum pathway 16, analysis port 18, negative pressure source 20, suction connector port 22, and/or other components of system 10. Cartridge body 12 is configured to contain the components of system 10 in a space small enough to be handheld and portable so system 10 may be easily transported within a medical facility, for example. In the example embodiment of system 10 shown in FIG. 1, cartridge body 12 has a length 100 running along a first axis 102 from a first side 104 to a second side 106 of about 30 mm to 60 mm. In some embodiments, cartridge body 12 may have a first width 108 near second side 106 running along a second axis 110 from a third side 112 to a fourth side 114 of about 15 mm to 40 mm. In some embodiments, cartridge body 12 may have a second width 116 near first side 104 running along second axis 110 from third side 112 to fourth side 114 of about 5 mm to 15 mm.

Cartridge body 12 has a first thickness 122 and a second thickness 124 running along a third axis 126 from a fifth side 128 toward a sixth side 130. In some embodiments, cartridge body 12 includes a first portion 118, wherein the thickness 122 of first portion 118 is substantially constant, and a second portion 120, wherein the thickness 124 increases relative to thickness 122. Thickness 122 may be about 3 mm to 10 mm. Thickness 124 increases toward second side 106. Thickness 124 may range from about 5 mm to about 30 mm. The general shape and approximate dimensions of cartridge body 12 shown in FIG. 1 and described herein are not intended to be limiting. Cartridge body 12 may take any shape that allows it to function as described in the present disclosure.

In some embodiments, cartridge body 12 is covered on sixth side 130 with a polyfilm cover 26. Polyfilm cover 26 is configured to cover sixth side 130 of system 10 such that open areas toward sixth side 130 in serum path 16, analysis port 18, negative pressure source 20, suction port 22, and/or other open areas on sixth side 130 are substantially sealed on sixth side 130 by polyfilm cover 26. Polyfilm cover 26 may be attached to cartridge body 12 with an adhesive and/or other methods of attachment. Polyfilm cover 26 may be configured with an opening 28 that corresponds to the location of filter 14 housed by cartridge body 12. Opening 28 is configured such that filter 14 may engage the quantity of blood.

Filter 14 is configured to separate blood plasma and/or serum from a quantity of blood. Filter 14 is attached to sixth side 130 of cartridge body 12. Filter 14 is configured with an entry side 30 and an exit side 32. Filter 14 is configured to engage an amount of blood on entry side 30 of filter 14. In some embodiments, filter 14 is configured to engage between about 20 μl and about 50 μl of blood. In some embodiments, filter 14 is configured to engage between about 20 μl and about 40 μl of blood. In some embodiments, filter 14 is configured to engage between about 20 μl and about 30 μl of blood. In some embodiments, filter 14 may be configured such that the amount of blood engaged by filter 14 permeates filter 14. Filter 14 may be configured with pores such that blood cells may be captured by filter 14 and blood plasma and/or serum is allowed to pass through filter 14 and exit filter 14 on exit side 32. In some embodiments, filter 14 may comprise filter paper, a filter membrane, and/or other filtering devices. By way of a non-limiting example, filter 14 may be a commercially available filter membrane.

In some embodiments, filter 14 and system 10 remain stationary and the amount of blood is disposed in proximity to filter 14 such that the blood engages filter 14. In some embodiments, the amount of blood remains stationary and filter 14 and system 10 are disposed in proximity to the amount of blood such that the blood engages filter 14.

In some embodiments, filter 14 may be configured with an external dimension 34 of between about 5 mm and 15 mm. In some embodiments, filter 14 may be configured with an external dimension of between about 6 mm and about 14 mm. In some embodiments, filter 14 may be configured with an external dimension of between about 8 mm and about 12 mm. External dimension 34 may be defined as a straight edge and/or a diameter. In FIG. 1, external dimension 34 is shown as a diameter. The circular shape of filter 14 shown in FIG. 1 is not intended to be limiting. In some embodiments, filter 14 may have a shape other than circular (e.g., rectangular, a square, an oval, etc.). In some embodiments, external dimension 34 of filter 14 may be optimized to engage between about 20 and 40 μl of blood.

In some embodiments, filter 14 may be mounted to cartridge body 12 with adhesive applied to the edges of filter 14. In some embodiments, filter 14 may be mounted to cartridge body 12 by ultrasound sealing.

Serum pathway 16 is configured to collect the separated blood plasma and/or serum at exit side 32 of filter 14. Serum pathway 16 is configured to transport the collected blood plasma and/or serum to analysis port 18. Serum pathway 16 is formed in cartridge body 12 near sixth side 130. Serum pathway 16 is configured to collect and transport the blood plasma and/or serum responsive to negative pressure in serum pathway 16 generated by negative pressure source 20, capillary forces generated by serum pathway 16, gravitational forces, and/or other forces. In some embodiments, serum pathway 16 comprises serum pit 40, a first channel 42, a second channel 44, and/or other components. Serum pit 40 is formed in cartridge body 12 such that filter 14 may be mounted over serum pit 40 with a sealing gasket (not shown). Serum pit 40 is positioned such that filtered blood plasma and/or serum collects in serum pit 40 after exiting filter 14 on exit side 32 of filter 14.

First channel 42 is configured to provide fluid communication between serum pit 40 and analysis port 18 such that the filtered blood plasma and/or serum is communicated from serum pit 40 to analysis port 18. First channel 42 is formed in cartridge body 12 near sixth side 130 substantially along first axis 102. In some embodiments first channel 42 has a triangular cross sectional shape oriented such that the deepest part of the channel is positioned away from filter 14 toward fifth side 128. In some embodiments first channel 42 may have a depth of between about 30 micrometers (μm) to about 300 μm. In some embodiments first channel 42 may have a depth of between about 40 μm to about 250 μm. In some embodiments first channel 42 may have a depth of between about 50 μm to about 200 μm. In some embodiments, one or more portions 46 of first channel 42 may extend into serum pit 40. In some embodiments, the blood plasma and/or serum flows from serum pit 40 through first channel 42 responsive to the negative pressure generated by negative pressure source 20, capillary forces generated by first channel 42, gravitational forces, and/or other forces. In some embodiments, first channel 42 may be hydrofilised.

Second channel 44 is configured to conduct negative pressure generated by negative pressure source 20, an external device connected to system 10 at suction connector port 22, and/or other sources through analysis port 18 to serum pit 40 such that the filtered blood plasma and/or serum is drawn into analysis port 18. Second channel 44 is formed in cartridge body 12 near sixth side 130 toward third side 112 substantially along first axis 102. In some embodiments second channel 44 may have a depth along third axis 126 of between about 30 micrometers (μm) to about 300 μm. In some embodiments second channel 44 may have a depth of between about 40 μm to about 250 μm. In some embodiments second channel 44 may have a depth of between about 50 μm to about 200 μm. As shown in FIG. 1, second channel 44 may be configured to jog around serum pit 40/filter 14 toward third side 112 to connect analysis port 18 to negative pressure source 20. Second channel 44 may be configured with the same triangular cross sectional shape as first channel 42, and/or second channel 44 may be configured with a cross sectional shape other than triangular.

Second channel 44 is configured to prevent the filtered blood plasma and/or serum from being drawn into negative pressure source 20. In some embodiments, cartridge body 12 may include a base (not shown in FIG. 1). The cartridge body 12 base may be configured to engage a substantially horizontal surface. Responsive to the base engaging the substantially horizontal surface, second channel 44 may ascend from analysis port 18 to negative pressure source 20. Second channel 44 may ascend to a level above the level of first channel 42 such that capillary forces in second channel 44 are reduced relative to capillary forces in other areas of serum pathway 16. Reduced capillary forces, gravitational forces, and/or other forces acting on the filtered blood plasma and/or serum in the ascending pathway provided by second channel 44 may limit and/or stop the flow of filtered blood plasma and/or serum such that the blood plasma and/or serum does not reach negative pressure source 20.

Analysis port 18 is configured to hold a quantity of plasma and/or serum during a plasma and/or serum analysis. Analysis port 18 is disposed in serum pathway 16 near sixth side 130 toward first side 104. Analysis port 18 is configured to provide an optical path for radiation from a radiation source associated with a spectroscope, for example, to pass through the plasma and/or serum during the plasma and/or serum analysis. Analysis port 18 is configured to be removably coupled with the spectroscope and/or other external analysis device such that a bilirubin level and/or other characteristics related to the filtered blood plasma and/or serum may be estimated. The estimation of the bilirubin level and/or other characteristics related to the filtered blood plasma/may be based on an analysis of the blood plasma and/or serum held by analysis port 18 performed by the spectroscope, for example.

In some implementations, cartridge body 12 may be configured to include features 50 such that analysis port 18 may be removably coupled to the external analysis device during the analysis of the blood plasma and/or serum contained in analysis port 18 (e.g., during a spectrophotometer analysis for bilirubin level). In some embodiments, features 50 of cartridge body 12 may be configured to maintain an orientation of analysis port 18 and/or cartridge body 12 relative to the analysis device such that second channel 44 ascends from analysis port 18 to negative pressure source 20 as described above. In FIG. 1, features 50 are located on first side 104, on third side 112 near first side 104, and on sixth side 130 near first side 104. The shapes and/or locations of features 50 are not intended to be limiting. Features 50 may take any shape and/or be located in any location in system 10 that allows system 10 to function as described herein.

In some implementations, the optical path provided by analysis port 18 may comprise an optical path window 52 formed in cartridge body 12. The radiation from the radiation source associated with the spectrometer, for example, may pass through optical path window 52. The radiation from the radiation source may pass through optical path window 52 on third side 112 near first side 104, through the blood plasma and/or serum held by analysis port 18, and/or through a corresponding window (not shown in FIG. 1) opposite optical path window 52 on fourth side 114 near first side 104. Optical path window 52 may comprise a one or more transparent areas of cartridge body 12 (e.g., 52 shown in FIG. 1 and the corresponding area not shown in FIG. 1 on fourth side 114 near first side 104), a separate transparent component coupled to cartridge body 12, and/or other components capable of conducting radiation. Analysis port 18 may be aligned with optical path window 52 of cartridge body 12. In some embodiments optical path window 52 may have an area of between about 1 square millimeter (mm²) and about 10 mm² In some embodiments, optical path window 52 may have an area of between about 1 mm² and about 7 mm². In some embodiments, optical path window 52 may have an area of between about 1 mm² and 5 mm².

Negative pressure source 20 is configured to generate a negative pressure in serum pathway 16 such that the filtered blood plasma and/or serum is drawn from exit side 32 of filter 14 into analysis port 18. Negative pressure source 20 is positioned near sixth side 130 toward second side 106 and third side 112 relative to filter 14. Negative pressure source 20 comprises a membrane 61 fixed to a cavity 60. Cavity 60 is formed in cartridge body 12 on fifth side 128 of membrane 61. In some embodiments, membrane 61 may comprise the portion of polyfilm cover 26 attached to sixth side 130 of cartridge body 12 positioned adjacent to cavity 60 of negative pressure source 20. In some embodiments, membrane 61 may be configured as a separate component attached to sixth side 130 of cartridge body 12.

Negative pressure is generated by negative pressure source 20 responsive to actuation of membrane 61. Membrane 61 is configured with a default position. Actuation of membrane 61 comprises deflection from and return to the default position by membrane 61. Membrane 61 is configured to deflect from the default position responsive to one or more forces acting on membrane 61. Membrane 61 is configured to return to the default position responsive to cessation of the one or more forces acting on membrane 61. In some implementations, actuation of membrane 61 may comprise finger pressure and release of the finger pressure by a user, for example.

Suction connector port 22 is configured to receive an external suction device. Examples of the external suction device may include a syringe and/or other external suction devices. The external suction device may optionally be engaged with suction connector port 22. The external suction device may be used instead of and/or in addition to negative pressure source 20 to generate negative pressure in system 10. Negative pressure source 20 may further comprise a cavity entry 62 and a cavity exit 64. Cavity exit 64 is connected to second channel 44. Cavity entry 62 is connected to suction connector port pathway 66. Suction connector port pathway 66 is configured to connect negative pressure source 20 to suction connector port 22. The negative pressure generated by the external suction device connected to suction port 22 may be conveyed through suction port 22, suction connector port pathway 66, cavity 60 of negative pressure source 20, and into serum pathway 16 such that filtered blood plasma and/or serum is drawn into analysis port 18.

In some embodiments, responsive to a deflection of membrane 61 from the default position, air is forced out through suction connector port 22 to the ambient environment. In some embodiments, suction connector port 22 further comprises a valve, and/or other airflow controlling device configured to substantially prevent and/or reduce air inflow through suction connector port 22 when membrane 61 returns to the default position.

FIG. 2 is a cross sectional view of system 10 taken through suction port 22, filter 14/serum pit 40, and analysis port 18. In FIG. 2, analysis port 18 is shown near first side 104. Filter 14 and serum pit 40 are shown near sixth side 130. Suction port 22 is shown at second side 106. In some embodiments, suction port 22 comprises a first column 200, a second column 202, and/or other components. First column 200 is configured to receive an external suction device. Second column 202 is configured to place the received external suction device in fluid communication with suction connector port pathway 66 (shown in FIG. 1) such that negative pressure generated by the external suction device is communicated to serum pathway 16 via column 202, suction connector port pathway 66 (shown in FIG. 1), and negative pressure source 20 (shown in FIG. 1). (As described relative to FIG. 1, polyfilm cover 26 is configured to seal second column 202 on sixth side 130. Second column 202 is configured to communicate with suction connector port pathway 66 via a connection on first side 104 of column 202 near sixth side 130.)

FIG. 3 illustrates an assembled view of system 10. Filter 14 and a sealing gasket 300 are assembled on sixth side 130 of cartridge body 12. Filter 14 is positioned adjacent to serum pit 40 (shown in FIG. 1, FIG. 2) on sixth side 130 of serum pit 40. Sealing gasket 300 is configured to hold filter 14 in position adjacent to serum pit 40.

FIG. 4 illustrates a method 400 for separating plasma and/or serum from blood with a plasma and/or serum separation system. The plasma and/or serum separation system comprises a filter, a serum pathway, an analysis port, and a negative pressure source. The operations of method 400 presented below are intended to be illustrative. In some embodiments, method 400 may be accomplished with one or more additional operations not described, and/or without one or more of the operations discussed. Additionally, the order in which the operations of method 400 are illustrated in FIG. 4 and described below is not intended to be limiting.

In some embodiments, method 400 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 400 in response to instructions stored electronically on an electronic storage medium The one or more processing devices may include one or more devices configured through hardware, firmware, and/or software to be specifically designed for execution of one or more of the operations of method 400.

At an operation 402, blood plasma and/or serum is separated from a quantity of blood with the filter. The filter is configured with an entry side and an exit side. In some embodiments, operation 402 is performed by a filter the same as or similar to filter 14 (shown in FIG. 1 and described herein).

At an operation 404, the separated blood plasma and/or serum is collected at the exit side of the filter with the serum pathway. In some embodiments, operation 404 is performed by a serum pathway the same as or similar to serum pathway 16 (shown in FIG. 1 and described herein).

At an operation 406, a quantity of plasma and/or serum is held during a plasma and/or serum analysis by the analysis port. The analysis port is disposed in the serum pathway. The analysis port is configured to provide an optical path for radiation to pass through the plasma and/or serum during the plasma and/or serum analysis. In some embodiments, operation 406 is performed by an analysis port the same as or similar to analysis port 18 (shown in FIG. 1 and described herein.)

At an operation 408, negative pressure is generated with the negative pressure source. The negative pressure is generated in the serum pathway such that the blood plasma and/or serum is drawn from the exit side of the filter into the analysis port with the combined forces of negative pressure and capillary forces. In some embodiments, operation 408 is performed by a negative pressure generator the same as or similar to negative pressure generator 20 (shown in FIG. 1 and described herein.)

In the claims, any reference signs placed between parentheses shall not be construed as limiting the claim. The word “comprising” or “including” does not exclude the presence of elements or steps other than those listed in a claim. In a system claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

1. A system for separating plasma and/or serum from blood, the plasma and/or serum separation system comprising:
a filter configured to separate blood plasma and/or serum from a quantity of blood, wherein the filter has an entry side and an exit side;
a serum pathway configured to collect the separated blood plasma and/or serum at the exit side of the filter;
an analysis port disposed in the serum pathway, configured to hold a quantity of plasma and/or serum during a plasma and/or serum analysis, wherein the analysis port is further configured to provide an optical path for radiation to pass through the plasma and/or serum during the plasma and/or serum analysis; and
a pressure source configured to communicate with the serum pathway such that at least a portion of the blood plasma and/or serum on the exit side of the filter is directed into the analysis port.

2. The system of claim 1, wherein the pressure source is a negative pressure source configured to generate a negative pressure in the serum pathway, wherein the negative pressure source comprises a membrane fixed to a cavity, and wherein the negative pressure is generated responsive to actuation of the membrane.

3. The system of claim 1, further comprising a base configured to engage a substantially horizontal surface, and wherein responsive to the base engaging the substantially horizontal surface during use of the system, the serum pathway ascends from the analysis port to the pressure source such that the flow of blood plasma and/or serum is prevented from flowing into the pressure source.

4. The system of claim 1, wherein the entry side of the filter has an external dimension of between about 5 and 15 millimeters.

5. The system of claim 1, wherein the analysis port is configured to be removably coupled to a spectroscope such that radiation emitted by a radiation source associated with the spectroscope travels through the blood plasma and/or serum in the analysis port along the optical path, thereby facilitating estimation of a bilirubin level in the blood plasma and/or serum based on an analysis by the spectroscope of the radiation that has traveled through the blood plasma and/or serum in the analysis port.

6. A method for separating plasma and/or serum from blood with a plasma and/or serum separation system, the plasma and/or serum separation system comprising a filter, a serum pathway, an analysis port, and a pressure source, the method comprising:
separating blood plasma and/or serum from a quantity of blood with the filter, wherein the filter has an entry side and an exit side;
collecting the separated blood plasma and/or serum at the exit side of the filter with the serum pathway;
holding a quantity of plasma and/or serum during a plasma and/or serum analysis with the analysis port, wherein the analysis port is disposed in the serum pathway, and wherein the analysis port is further configured to provide an optical path for radiation to pass through the plasma and/or serum during the plasma and/or serum analysis; and
generating a pressure, with the pressure source, in the serum pathway such that at least a portion of the blood plasma and/or serum on the exit side of the filter is directed into the analysis port by the combined forces of pressure and capillary forces.

7. The method of claim 6, wherein the generated pressure is a negative pressure, wherein the negative pressure is generated responsive to actuation of a membrane, and wherein the pressure source comprises the membrane fixed to a cavity.

8. The method of claim 6, further comprising preventing the flow of blood plasma and/or serum from flowing into the pressure source with the serum pathway, wherein the system further comprises a base configured to engage a substantially horizontal surface, and wherein responsive to the base engaging the horizontal surface during use of the system, the serum pathway ascends from the analysis port to the pressure source such that the flow of blood plasma and/or serum is prevented from flowing into the pressure source.

9. The method of claim 6, wherein the entry side of the filter has an external dimension of between about 5 and 15 millimeters.

10. The method of claim 6, further comprising removably coupling the system to a spectroscope with the analysis port such that radiation emitted by a radiation source associated with the spectroscope travels through the blood plasma and/or serum in the analysis port along the optical path, thereby facilitating estimation of a bilirubin level in the blood plasma and/or serum based on an analysis by the spectroscope of the radiation that has traveled through the blood plasma and/or serum in the analysis port.

11. A system for separating plasma and/or serum from blood, the plasma and/or serum separation system comprising:
means to separate blood plasma and/or serum from a quantity of blood, wherein the means to separate has an entry side and an exit side;
means to convey blood plasma and/or serum, the means to convey configured collect the separated blood plasma and/or serum at the exit side of the means to separate;
means, disposed in the means to convey, to hold a quantity of plasma and/or serum during a plasma and/or serum analysis, wherein the means to hold is further configured to provide an optical path for radiation to pass through the plasma and/or serum during the plasma and/or serum analysis; and
means to generate a pressure in the means to convey, the means to generate configured to communicate with the means to convey such that at least a portion of the blood plasma and/or serum on the exit side of the means to separate is directed into the means to hold.

12. The system of claim 11, wherein the means to generate generates a negative pressure, wherein the means to generate comprises a membrane fixed to a cavity, and wherein the negative pressure is created responsive to actuation of the membrane.

13. The system of claim 11, further comprising means to engage a substantially horizontal surface, and wherein responsive to the means to engage engaging the substantially horizontal surface during use of the system, the means to convey ascends from the means to hold to the means to generate such that the flow of blood plasma and/or serum is prevented from flowing into the means to generate.

14. The system of claim 11, wherein the entry side of the means to separate has an external dimension of between about 5 and 15 millimeters.

15. The system of claim 11, wherein the means to hold is configured to be removably coupled to a spectroscope such that radiation emitted by a radiation source associated with the spectroscope travels through the blood plasma and/or serum in the means to hold along the optical path, thereby facilitating estimation of a bilirubin level in the blood plasma and/or serum based on an analysis by the spectroscope of the radiation that has traveled through the blood plasma and/or serum held by the means to hold.
